(19) United States
(12) Patent Application Publication
(10) Pub. No.: US 2025/0207301 A1
(43) Pub. Date: Jun. 26, 2025

(54) ARTICLE OF CLOTHING FOR PROTECTING AN AFFECTED AREA OF THE SKIN

(71) Applicant: Barbara DESCLAUX, PARIS (FR)

(72) Inventor: Barbara DESCLAUX, PARIS (FR)

(21) Appl. No.: 18/988,271

(22) Filed: Dec. 19, 2024

(30) Foreign Application Priority Data
Dec. 26, 2023 (FR) 2315282

(51) Int. Cl. D02G 3/38; A61M 35/00; D02G 3/32; D02G 3/36

(52) U.S. Cl. CPC D02G 3/38; A61M 35/10; D02G 3/328; D02G 3/36; D10B 2201/24; D10B 2211/04; D10B 2501/00; D10B 2509/00

(57) ABSTRACT

An article of clothing intended to be worn by a subject in contact with the skin, the article including at least one composite yarn or a first of composite yarns, the composite yarn including an elastic central core covered with a sheath composed of cellulosic fibers.

### CROSS-REFERENCE TO RELATED APPLICATIONS

This application claims priority to French Patent Application No. 2315282, filed Dec. 26, 2023, the entire content of which is incorporated herein by reference in its entirety.

### FIELD

The invention relates to an article of clothing, notably intended to cover an area of affected skin, preferably of the atopic type such as atopic dermatitis.

### BACKGROUND

Eczema, a common and often chronic skin disorder, affects millions of people around the world. Symptoms such as itching, redness, and skin irritation can significantly alter the quality of life of the affected subjects.

Currently, many skincare products require the user to patiently wait for the applied cream to dry, thereby limiting their ability to dress or handle objects during this period. This time constraint can represent a significant obstacle in the daily routine of users, leading to loss of time and efficiency, as well as a potential decrease in user satisfaction.

In today's textile industry, the widespread use of unsuitable materials often compromises the natural hygiene of the skin. Among these materials, synthetic fibers occupy a prominent place, frequently incorporated into sportswear due to their ability to dry quickly. Unfortunately, these synthetic fibers, whether used pure or mixed with other materials, also dominate the composition of ready-to-wear garments. This prevalence poses a major challenge for subjects with sensitive skin, in particular those with eczema.

One of the main concerns related to the use of synthetic fibers resides in their tendency to irritate sensitive skin. Indeed, when a person sweats, these fibers promote bacterial proliferation due to their inability to absorb moisture released from the body. This accumulation of moisture on the skin's surface creates an environment conducive to maceration, compromising skin comfort and hygiene. In addition, synthetic fibers, as inert elements, do not contribute favorably to the environment necessary for the skin's natural regeneration.

Documents US2013/131,563, CN112603270A, CN106192177A, CN209873231U describing elastic yarns for making articles of clothing are known. However, none of these documents are suitable for skin suffering from eczema.

The textile materials commonly used in the manufacture of garments, in particular synthetic fibers, do not adequately meet the needs of subjects with sensitive skin, in particular those with eczema. The main shortcoming identified is the inability of these synthetic fibers to effectively manage body moisture, thereby promoting bacterial proliferation and compromising skin integrity. It becomes imperative to develop an innovative solution that mitigates these problems inherent to synthetic fibers, thus offering a textile garment that actively supports the health and well-being of subjects suffering from skin sensitivities, while promoting an environment conducive to epidermal regeneration.

### SUMMARY

One or more aspects of the invention aim to provide an article of clothing intended to be worn on the surface of a subject's skin overcoming the limitations of the prior art.

According to a first aspect, the invention relates to an article of clothing intended to be worn by a subject in contact with the skin comprising at least one composite yarn or a first group of composite yarns; the composite yarn comprising an elastic central core covered with a sheath composed at least of cellulosic fibers.

A benefit is to provide a compressive article of clothing in which the elastic yarns cannot be in contact with the subject.

In an embodiment, the sheath of the first composite yarn or the first group of yarns completely covers the elastic central core.

In an embodiment, cellulosic fibers of the sheath comprise a viscose or a viscose derivative.

In an embodiment, the first composite yarn is a gimped yarn formed of the elastic central core and at least one first covering yarn, made of cellulosic fiber material, gimped around the elastic central core and forming the sheath.

In an embodiment, the first composite yarn further comprises at least one second covering yarn gimped around the central core.

In an embodiment, the second covering yarn is a silk yarn.

In an embodiment, the elastic central core of the first composite yarn or the first group of composite yarns comprises an elastane yarn.

In an embodiment, said article is obtained by knitting the first composite yarn or the first group of yarn with a second silk yarn or a second group of silk yarns.

In an embodiment, the first composite yarn and the second yarn have, relative to each other, a defined position such that the second yarn or the second group of yarn only is visible on a first face of the article.

In an embodiment, the first face of the article is the inner face of the article intended to be in contact with the skin of a subject when the article of clothing is worn by said subject.

In one embodiment, the article is composed of, or consists of, between 20% and 40% of the first yarn or the first group of yarns and between 60% and 80% of the second yarn or the second group of yarns.

In an embodiment, the article is obtained by knitting the first yarn or the first group of yarn with a second yarn or a group of yarn.

In an embodiment, the article is designed to form a compressive garment intended to be worn by a subject.

In an embodiment, the article of clothing does not comprise a seam (i.e. it is free of a seam or provided without a seam). In an embodiment, the article does not comprise a button and/or zipper-type closure (i.e. it is free of a button and/or zipper-type closure or provided without a button and/or zipper-type closure).

According to a second aspect, the invention relates to a method of therapeutic treatment of an affected area of the skin of a subject comprising the following steps: providing an article according to an embodiment of the invention and placing the article on the subject so as to fully cover the affected area.

According to an embodiment, the method further comprises the wearing of the placed article for at least a predetermined duration.

According to an embodiment, wherein the predetermined duration is at least 1 h.

According to an embodiment, wherein the predetermined duration is at least 6 h or 8 h.

According to an embodiment, the method further comprises a step of cleaning the affected area before the placement step.

According to an embodiment, the method further comprises the application of a medicinal cream or a medicinal preparation on said affected area of the subject.

According to an embodiment, the medicinal cream comprises a cream for the treatment of atopic dermatitis or eczema such as a cream comprising a corticosteroid, also called corticosteroid cream.

### BRIEF DESCRIPTION OF THE FIGURES

FIG. 1 shows a schematic representation of a composite yarn according to an embodiment of the invention comprising two covering yarns wound around an elastic central core in a disjointed manner.

FIG. 2 shows a schematic representation of a composite yarn according to an embodiment of the invention comprising a single covering yarn wound around an elastic central core in a disjointed manner.

FIG. 3 shows an example of a two-dimensional knitting structure of an article according to an embodiment of the invention wherein a second silk yarn is used as knit and the composite yarn is used as weft yarn for one out of two rows of the knit.

FIG. 4 shows a schematic cross-sectional view of an article of clothing according to an embodiment of the invention wherein the article is designed such that at least one face is designed with a layer of second yarn.

FIG. 5 shows a schematic view of a subject wearing an article of clothing according to an embodiment of the invention wherein the article is an elbow protector.

FIG. 6 is a flowchart representing the steps of a method according to an embodiment of the invention.

### DETAILED DESCRIPTION

The aforementioned technical problem is solved in accordance with an aspect of the invention thanks to a composite yarn 10 comprising an elastic central core 11 covered with a sheath composed of cellulosic fibers. In an embodiment, cellulosic fibers are taken to mean plastics of plant origin obtained by subjecting the cellulose to chemical and physical treatments aimed at dissolving and shaping it. The elastic central core 11 beneficially provides the article of clothing 1 with elastic properties thus making it possible to design compressive garments intended to adhere to the skin of the subject 30.

The sheath composed of cellulosic fibers makes it possible to avoid contact between the elastic central core 11 and the skin of the subject 30 and also allows for rapid absorption of excess moisture, reducing bacterial proliferation on the affected areas.

“Article of clothing” means all or part of a garment intended to be worn by a subject 30.

The article of clothing 1 may comprise all or part of an article 1 such as, as a non-limiting example, an underwear, a hat, a sock, a singlet, a cuff, an elbow pad, a leg piece, tights, a hood, a pajama, gloves, mittens.

FIG. 5 shows a subject 30 clothed with an elbow protector-type article of clothing 1, particularly beneficial if the subject 30 has an affected area at the elbow or elbow fold.

In an embodiment, the article of clothing 1 is all or part of a compressive garment.

“Compressive garment” means a garment that is elastic and stretchable in all directions that locally exerts continuous pressure on the surface of the skin of the subject 30 wearing the garment. The pressure is exerted continuously by the garment when it is worn by the subject 30.

The compressive aspect is beneficially provided by the elastic central core 11 of the composite yarn 10.

### Composite Yarn

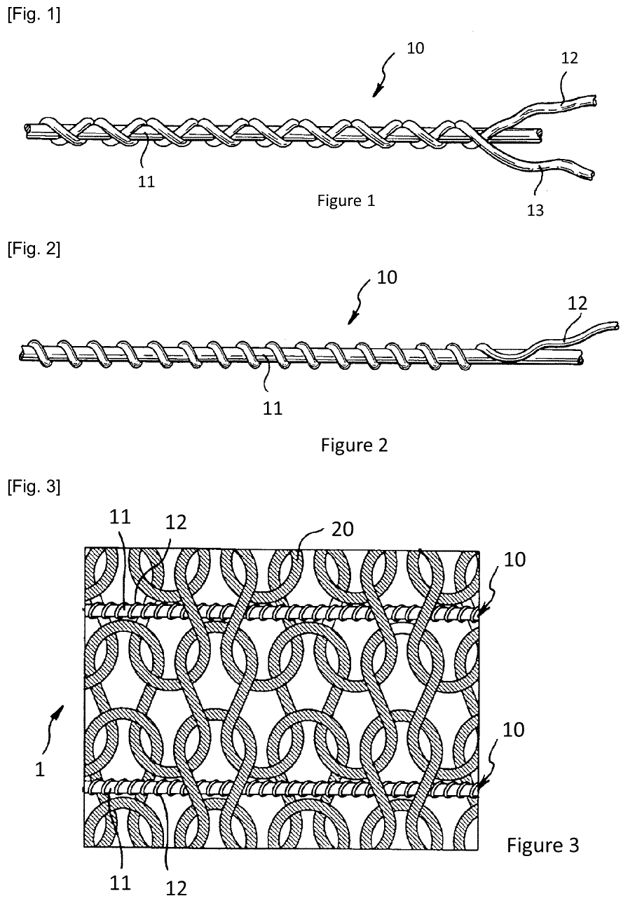

An example of composite yarn 10 intended to constitute at least a part of an article of clothing 1 in accordance with the invention is described below with reference to FIGS. 1 and 2.

In these examples, the composite yarn 10 is a gimped yarn. “Gimped yarn” means a yarn formed by the regular helix winding of one or more covering yarns 12, 13 or around another yarn (the central core), desirably by covering it completely, i.e. the windings are present over the entire length of the central core or partially, i.e. the windings are present over a portion of the length of the central core. In one embodiment, the successive windings of the covering yarns around the core are then jointed. The jointed windings then cover the entire surface of the central core over all or part of the length of the yarn by contact between two successive turns. In another embodiment, the successive windings are said to be “disjointed” when the successive turns are not in contact with each other, leaving the central core appearing.

The composite yarn 10 is a yarn composed of at least two different materials. The composite yarn 10 comprises an elastic central core 11 and at least one covering yarn 12, 13 forming the sheath.

In FIG. 1, the composite yarn 10 comprises 2 covering yarns 12, 13 wound in opposite helices, in accordance with a double gimping technique. In FIG. 2, an alternative of a composite yarn 10 including a single covering yarn 12 is shown, in accordance with a single gimping technique.

The covering yarn(s) 12, 13 are gimped around the elastic central core 11 by spiral winding around the elastic central core 11. Other alternatives of non-represented gimping technique such as gimping by entanglement may be envisaged.

In an embodiment, the covering yarns 12,13 are wound so as to establish direct contact with the elastic central core 11.

For reasons of clarity, FIGS. 1 and 2 show the covering yarn(s) 12, 13 covering the elastic central core 11 with “disjointed” turns: the successive windings of the covering yarn(s) 12, 13 around the elastic central core 11 are disjointed and are not in contact with each other, leaving the elastic central core 11 appearing.

In an embodiment, the covering yarn(s) 12, 13 completely cover the elastic central core 11 and the successive windings of the covering yarn(s) 12, 13 around the elastic central core 11 are joined. Each turn is in contact with the previous turn and the next turn.

A benefit of an embodiment where the covering yarns 12, 13 fully cover the elastic central core 11 is to ensure that the elastic central core 11 will not be in contact with the subject's skin.

The yarn(s) constituting the elastic central core 11 could be made of elastane, or any other material having similar mechanical properties.

The covering yarn(s) 12, 13 are, in an embodiment, composed of cellulosic fibers.

In an embodiment, the covering yarn(s) 12, 13 are composed of (or consist(s) of) viscose or a viscose derivative. For example, the covering yarn(s) 12, 13 are composed of Lyocell. Lyocell is a viscose produced from wood pulp, notably eucalyptus. The viscose beneficially allows effective absorption and quick drying.

The covering yarn(s) 12,13 may also be composed of (or consist(s)) any other fiber or material capable of absorbing moisture.

In an embodiment, the covering yarn(s) 12, 13 comprise a mixture of cellulose fibers with an animal fiber. The animal fiber may comprise chitin. The chitin may be extracted from crab shell. It may be reduced in powder form then spun by a method similar to the viscose manufacturing process then mixed with cellulosic fibers in a variable proportion (from 1% m to 99% m) to form the covering yarn.

Such a covering fiber comprising a mixture of chitin fibers and cellulosic fibers (such as lyocell) is particularly beneficial in that it comprises a strong moisture-absorbing power and is an allergic. The covering of the elastic central core 11 by a sheath of cellulosic fiber, notably lyocell, beneficially allows good moisture absorption, such a sheath allows good mechanical resistance of the article of clothing 1.

Notably, the combination of an elastane central core 11 and a sheath of cellulosic fibers of the lyocell type has demonstrated good elasticity properties enabling compressive articles of clothing to be produced, on the one hand, good moisture absorption and a quick-drying ability, notably superior to cotton and silk, providing an article of clothing with good antibacterial properties, as well as low abrasion with the skin. These benefits allow for better comfort when said article of clothing is worn on an affected area, notably an area of the skin subject to atopic dermatitis (or eczema).

In an embodiment wherein a plurality of covering yarns 12, 13 are wound around the elastic central core 11, one of the covering yarns may be comprised of a silk protein fiber yarn (referred to as “silk yarn” in the remainder of the description).

In an embodiment, the silk yarn is a yarn consisting only of silk protein fibers. In an embodiment, the silk yarn is a yarn consisting of at least 98% m silk protein fibers.

In an embodiment, the cellulosic fiber yarn is a yarn consisting only of cellulosic fibers. In an embodiment, the cellulosic fiber yarn is a yarn consisting of at least 98% m cellulosic fibers.

In an embodiment, the covering yarns comprise a first covering yarn 12 made of cellulosic fibers and a second covering yarn 13 made of silk.

The silk provides the article of clothing 1 with soft, non-slip texture properties, unlike synthetic fibers. This beneficially makes it possible to reduce the abrasive aspect of the article of clothing 1 against the affected surface of the subject's skin. The use of silk is beneficial for its lubricating (anti-abrasive), thermoregulating (to regulate moisture in contact with the skin) and lipophobic (to avoid absorbing a cream applied to the subject's skin) properties.

### Article of Clothing

In an embodiment, the article of clothing 1 comprises at least one portion made from the composite yarn 10 as described above.

In an embodiment, the portion is made by knitting of the composite yarn 10. The portion may be made entirely from the composite yarn 10.

In an embodiment, the portion of the article of clothing 1 is made from a composite yarn 10 or a group of composite yarn 10 as described above and from a second yarn or a second group of yarn. In an embodiment, the second yarn 20 is a silk yarn.

The use of silk is beneficial for its biocompatibility, lubricating (anti-abrasive), thermoregulating (to reduce moisture in contact with the skin) and lipophobic (to prevent absorption of a cream applied to the subject's skin) properties.

In an example illustrated in FIG. 3, the portion of article of clothing 1 is made by knitting of the second yarn 20. The weft yarn, which is inserted into the knit made with the second yarn 20, consists of a composite yarn 10 as described above. In an embodiment, the weft yarn is inserted into the knit formed by the second yarn 20 without entering into the stitch of said knit.

In this case, a weft yarn is provided for every second row of the knit. This example is non-limiting and one weft yarn may be provided for each row or for one row out of three or out of four.

The gimped composite yarn 10 shown in FIG. 3, for simplicity of representation, has been shown with a single disjointed covering yarn 12 wound around the elastic central core 11, but any composite yarn 10 described above may be envisaged, for example a gimped yarn with two covering yarns and/or jointed turns.

Finally, still for the sake of simplicity of representation, a 2D knitting has been represented in FIG. 3, but it is understood that the portion of article of clothing 1 may comprise any type of three-dimensional knitting.

In an embodiment, the portion of article of clothing 1 comprises between 60% and 80% of the second yarn 20 or group of second yarns 20 such as silk yarn and comprises between 20% and 40% of composite yarn 10.

The percentages are expressed in yarn distance in the article 1.

In an embodiment, the article of clothing does not comprise seams or button or zip type closure means. In an embodiment, the thickness of the article of clothing is constant along the length and width of said article. By constant, it is ideally understood that the article comprises a number of stitches of constant thickness over the entire surface of the article.

### Plaiting

In an embodiment, the portion of article of clothing 1 comprises a plaiting on one of these faces, such as the face intended to be in contact with the skin of the subject.

Plaiting is a knitting method known to those skilled in the art in which two different yarns are knitted together so that one covers the other. In one embodiment, the composite yarn 10 and the second yarn 20 are knitted so that the second yarn 20 covers the first composite yarn 10 on the surface of the article of clothing 1.

In an embodiment, the composite yarn 10 is used on one face with the needles in front needle beds which position it at the front of the knit and the second yarn such as a silk yarn is knitted with the rear needle beds, thus the yarns are brought back to the rear face of the knit or vice versa. In one embodiment, the composite yarn is used in ribbing, i.e. it is knitted together with the second yarn in a partial manner, i.e. one stitch out of two, one stitch out of three, or less.

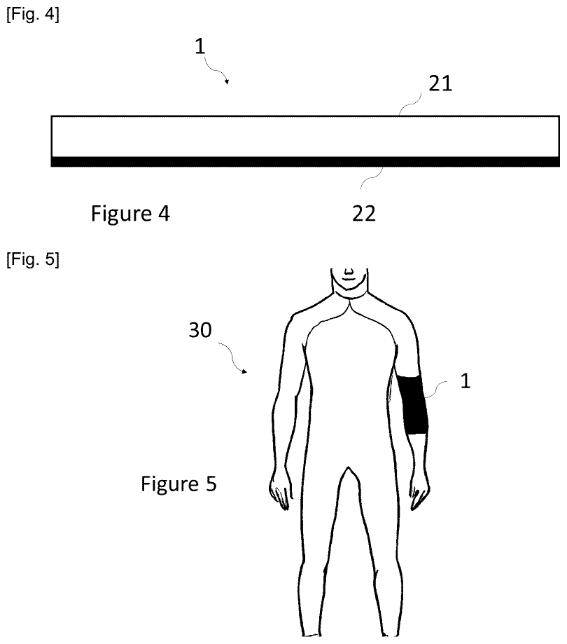

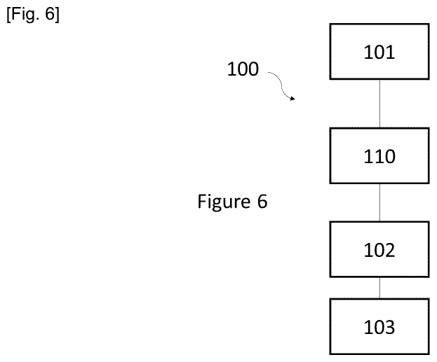

Generally, as illustrated in FIG. 4, the portion comprises a first face 21 as described above and a second face 22 made so that the skin of the subject in contact with the second face 22 is in contact only with the second yarn 20 or the second group of yarns.

A benefit, when the second yarn is a silk yarn, is to improve the subject's comfort at the affected area, notably when the affected area is a very dry eczema, while retaining the moisture absorption and elasticity properties of the composite yarn 10.

Another benefit is to allow the application of a medicinal cream for the treatment of the affected area and the placement of the article of clothing 1 on the area. Indeed, silk fibers, thanks to their lipophobic properties, will exhibit reduced absorption of the cream, notably compared to fibers such as lyocell or cellulosic fibers.

Other knitting or design techniques may be envisaged so as to obtain an article of clothing 1 comprising a face composed of the second yarn.

In an embodiment, the article of clothing 1 is designed to be reversible, for example so that by turning it, the first face 21 is then in contact with the subject's skin. A benefit is to have a single article of clothing 1 to be used either to have its surface more lubricant, thermoregulating and lipophobic (second face 22), or to have a more absorbent surface (first face 21) in contact with the skin of the subject wearing the article of clothing 1. The improvement of the absorbency of the first face 21 is particularly beneficial when the subject sweats, for example during physical exertion, or when the affected area has pruriginous discharges or exudates.

In an embodiment, the article of clothing 1 or the portion of article of clothing 1 is made of a single seamless textile piece. The seamless textile beneficially makes it possible to reduce discomfort due to seams on the skin.

### Therapeutic Method

Another aspect of the invention consists of a therapeutic method 100 for treating an affected area.

An affected area may be understood as an area of the surface of the skin having at least one lesion. In an embodiment, a lesion comprises atopic dermatitis. In an embodiment, said lesion may comprise a skin burn and/or any change produced on the skin by a disease or an accident.

The therapeutic method comprises the use 110 of a therapeutic article 1 according to an embodiment of the invention to cover the affected area. “Affected area” means an area of the surface of the skin of the subject comprising at least one atopic lesion as previously described.

In an embodiment, the method comprises a step of cleaning (110) the affected area. The cleaning may be done with any type of cleaning product suitable for cleaning or rinsing the affected area such as water, a disinfectant product, shower oil. In an embodiment, the cleaning step is carried out without soap or product derived from saponification. In an embodiment, the method comprises the application 120 of a cream or a drug solution on the affected area of the skin before covering the area by the article of clothing 1. The cream or drug solution, in an embodiment, comprises a moisturizing cream, a cream comprising a corticosteroid, or any drug allowing the treatment of dermatitis such as atopic dermatitis.

The article of clothing 1 is then put on 130 so as to be worn by the subject so that the article 1 (for example its second face 22) covers the affected area, in an embodiment, in full.

In an embodiment, the article of clothing 1 is then worn by the subject for at least a predetermined duration.

The predetermined duration may be at least 1 h. A benefit is to allow the application of the drug in the subject's epidermis.

In an embodiment, the article of clothing 1 is worn by the subject for a minimum duration of 6 h or 8 h. The benefit is to make it possible to obtain the beneficial effects of compression and the non-abrasive surface on the affected area as well as the antibacterial properties of the article 1 according to the invention.

### General Benefits

The article 1 according to an embodiment the invention beneficially makes it possible to compress the affected area of the subject. The compression initially enables a feeling of soothing by offering a constant sensation of body limit as well as a feeling of pressure. In addition, such compression reduces damage from scratching by the subject on the affected area. The wearing of a compressive article 1 thus enables the reduction of the signs (pain, itching, pruritus, erythema) of the inflammation.

Another benefit is the non-contact between the skin and the synthetic fibers, reducing the abrasion of the affected areas with the garment. In addition, the article of clothing 1 and the silk face beneficially allow the application of cream on the affected area that will be covered thanks to the low absorption property of the silk. A benefit is to promote contact between the cream and the affected area of the subject by reducing the risk of absorption of the cream by the garments. In addition, such an article 1 makes it possible to reduce the risk of removal of the cream by contact of the affected area by contact with another part of the body or an object. This aspect is particularly beneficial when the article of clothing 1 is a glove for covering an affected area on the hand. The subject can then move or handle objects after applying a cream without fear that the cream will be deposited on the handled objects.

The article of clothing 1 according to an embodiment of the invention thus also makes it possible to overcome the drawbacks of applying a cream (drying time, time constraint) by proposing a device allowing a more efficient application of cream and adapted to the constraints of daily life.

Finally, the article of clothing 1 beneficially makes it possible to combine the aspects of non-abrasive surface, elasticity and moisture absorption and removal at the surface of the skin.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements, components, compositions, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. An article of clothing intended to be worn by a subject in contact with the skin, the article comprising at least one composite yarn or a first group of composite yarns, said composite yarn comprising an elastic central core covered with a sheath composed at least of cellulosic fibers.

2. The article according to claim 1, wherein the sheath of the first composite yarn or of the first group of yarns completely covers the elastic central core.

3. The article according to claim 1, wherein the cellulosic fibers of the sheath comprise a viscose or a viscose derivative.

4. The article according to claim 1, wherein the first composite yarn is a gimped yarn formed
of an elastic central core, and
of at least one first covering yarn, made of cellulosic fiber material, gimped around the elastic central core and forming the sheath.

5. The article according to claim 4, wherein the first composite yarn further comprises at least one second covering yarn gimped around the central core.

6. The article according to claim 5, wherein the second covering yarn is a silk yarn.

7. The article according to claim 1, wherein the elastic central core of the first composite yarn or of the first group of composite yarns comprises an elastane yarn.

8. The article according to claim 1, wherein said article is obtained by knitting of the first composite yarn or the first group of yarn with a second silk yarn or a second group of silk yarns.

9. The article according to claim 8, wherein the first composite yarn and the second yarn have a defined position relative to one another, such that the second yarn or the second group of yarn only is visible on a first face of the article.

10. The article according to claim 9, wherein the first face of the article is the inner face of the article intended to be in contact with the skin of a subject when the article of clothing is worn by said subject.

11. A method of therapeutically treating an affected area of the skin of a subject, the method comprising providing an article according to claim 1 and placing the article on the subject so as to fully cover the affected area.

12. The method according to claim 11, further comprising applying a medicinal cream or a medicinal preparation on said affected area of the subject.

13. The method according to claim 12, wherein said medicinal cream comprises a cream for the treatment of atopic dermatitis or eczema.

14. The method according to claim 13, wherein the cream comprises a corticosteroid.

15. The method according to claim 11, further comprising wearing said placed article for at least a predetermined duration.

16. The method according to claim 15, wherein the predetermined duration is at least 1 h.

17. The method according to claim 16, wherein the predetermined duration is at least 6 h or 8 h.
